(19) United States
(12) Patent Application Publication
(10) Pub. No.: US 2007/0233031 A1
(43) Pub. Date: Oct. 4, 2007

(54) Package for folded absorbent articles

(76) Inventors: William Mercer Benson, Harrison, OH (US); Alan Edwin Ineson, Raymond, ME (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/391,153

(22) Filed: Mar. 28, 2006

(51) Int. Cl. A61F 13/15

(52) U.S. Cl. 604/385.02

(57) ABSTRACT

A package of absorbent articles is disclosed. The package includes a container made of flexible sheet material, and a line of weakness defining a predetermined portion of the container to be separated from the container. The container has a front panel and at least a portion of the line of weakness is located within the front panel of the container. The predetermined portion has an opening dimension. The package includes a plurality of pouches arranged in a stacked relationship within the container. The pouch transverse edge width and pouch longitudinal edge width can be greater than the opening dimension.

### FIELD OF THE INVENTION

The present invention relates to a package for absorbent articles.

### BACKGROUND OF THE INVENTION

Retailers commonly sell absorbent articles to consumers in containers in which more than one absorbent article is enclosed. Some containers may contain as many as twenty or more absorbent articles. The consumer must open the container to retrieve an absorbent article from the container.

Often, the consumer needs to immediately use the absorbent article and does not have the luxury of acquiring a suitable implement to neatly open an unopened container. If the container does not have an opening mechanism, such as line of weakness in the container or a removable seal, the consumer is left with the option of tearing open the container. If the container is comprised of flexible sheet material, the opening created by the consumer may not perform as desired. For instance, if the opening is too small, the consumer may have difficulty reaching into the container to grasp a single absorbent article. If the opening is too large, the consumer can easily grasp a single absorbent article but the remaining absorbent articles can spill out of the container. Furthermore, if the consumer tears open the container, the consumer may mar the product description and branding identity on the exterior of the container. Consumer products companies expend a tremendous amount of effort to communicate with the consumer and to build brand identity for their wares to help consumers make purchasing decisions. These efforts can be defeated by a container that performs poorly.

Manufacturers of absorbent articles often place individual absorbent articles within a pouch to prevent contamination of the absorbent article prior to use. For instance, panti-liners and sanitary napkins are commonly packaged in a pouch so that the user can obtain the article from the container and carry the article around in her pocket or purse until she needs to use the article. An absorbent article may be placed in a pouch in a folded configuration, with all or part of the absorbent article enclosed in the pouch. Different edges of the pouch may have different contours and thicknesses and some edges may be easier for the consumer to grasp than others, depending on the geometry of the edge of the pouch.

A typical consumer generally uses only one absorbent article at a time. To obtain an absorbent article from a container containing multiple absorbent articles, the consumer holds the container in one hand and uses her thumb and forefinger of her other hand to grasp a single absorbent article. Conventional containers for absorbent articles do not address the complicated ergonomics of the consumer's action. Many containers for absorbent articles are too large to be easily grasped with one hand. When some containers are grasped, multiple absorbent articles can be discharged through the opening of the container. In other containers, absorbent articles that are enclosed in a pouch can be difficult for the consumer to retrieve because an edge of the pouch that can be easily grasped is not presented to the consumer through the opening of the container.

Consumers often leave absorbent articles within the container and store the container in a drawer or closet after opening the container. Often, the location where the container is stored is used for other household purposes. As the consumer and her family move about their daily lives, the containers may be deformed, scrunched, tipped over, knocked off of the shelf on which the container is stored, or dropped by the consumer. Many containers for absorbent articles that are presently in the marketplace perform unsatisfactorily during storage because the absorbent articles contained therein spill from the container during storage.

With these limitations in mind, there is a continuing unaddressed need for packages of absorbent articles that are easy to open and preserve the appearance of the container after opening. There is a further continuing unaddressed need for packages of absorbent articles that address the ergonomics associated with enabling a consumer to easily retrieve a single absorbent article from a container. There is also a continuing unaddressed need for packages of absorbent articles that when opened permit the consumer to retrieve a single absorbent article and additional absorbent articles do not easily spill out of the container during storage.

### SUMMARY OF THE INVENTION

A package of absorbent articles is disclosed. The package includes a container having a front panel having a front panel width, a back panel opposing the front panel, opposing side panels, a top panel having a top panel width, and a bottom panel opposing the top panel, which are joined together to form an interior compartment. The panels comprise flexible sheet material. The package includes a line of weakness, wherein at least a portion of the line of weakness is located within the front panel. In one embodiment, the line of weakness defines a predetermined portion of the container to be separated from the container. The predetermined portion comprises an opening dimension. The package includes a plurality of pouches arranged in a stacked relationship in the interior compartment of the container, each pouch comprising a pouch longitudinal centerline and a pouch transverse centerline, a pair of pouch longitudinal edges generally disposed along the pouch transverse centerline and a pair of pouch transverse edges generally disposed along the pouch longitudinal centerline, a pouch longitudinal edge length defined by the distance between the pair of pouch transverse edges, and a pouch transverse edge width defined by the distance between the pair of pouch longitudinal edges. The pouch longitudinal edge length and the pouch transverse edge width can be greater than the opening dimension. The package includes a plurality of folded absorbent articles, wherein at least one folded absorbent article is contained in each pouch. In an alternative embodiment, the line of weakness defines an opening slit.

In one embodiment, a portion of the line of weakness is located in the top panel. In another embodiment, the pouch transverse edge width is more than about two times greater than the opening dimension. In another embodiment, one of the pouch transverse edges is substantially facing the front panel and substantially parallel to the top panel. In another embodiment, one of the pouch transverse edges is substantially facing the top panel and substantially parallel to the front panel. In another embodiment, the folded absorbent article is bi-folded or tri-folded. In another embodiment, the pouch has an aspect ratio defined by the pouch longitudinal edge length divided by the pouch transverse edge width, wherein the aspect ratio is between about 0.5 and about 1.2. In another embodiment, the container contains between about 10 and about 40 pouches arranged in a single stack. In another embodiment, the container contains between about 30 and about 100 pouches arranged in two or more stacks, each stack comprising an approximately equal number of pouches. In one embodiment, the absorbent articles are selected from the group consisting of sanitary napkins, panti-liners, and incontinence products. In one embodiment, the bottom panel has a bottom panel surface area and a bottom panel largest edge length, wherein the bottom panel surface area is less than about 7300 square millimeters and the bottom panel largest edge length is less than about 90 mm. In one embodiment, the absorbent article comprises a liquid pervious topsheet, a backsheet joined to the topsheet, and an absorbent core positioned between the topsheet and the backsheet.

### BRIEF DESCRIPTION OF THE DRAWINGS

FIG. 1 is a cut-away view of a package of absorbent articles in which the line of weakness is an opening slit.

FIG. 2 is a cut-away view of a package of absorbent articles in which a portion of the line of weakness is on the front panel and a portion of the line of weakness is on the top panel.

FIG. 3 illustrates a container in which the front panel line of weakness length is about the same as the top panel line of weakness length.

FIG. 4 is a cut-away view of a package of absorbent articles in which the predetermined portion resides entirely on the front panel of the container.

FIG. 5 illustrates a container in which the predetermined portion is separated from the container.

FIG. 6 illustrates a consumer using her thumb and forefinger of one hand to remove a pouch from the container.

FIG. 7 illustrates the components of a pouch.

FIG. 8 illustrates a partially opened pouch that does not comprise a flap panel.

FIG. 9 illustrates a partially opened pouch that comprises a flap panel.

FIG. 10 illustrates a pouch that does not comprise a flap panel and comprises a pull tab.

FIG. 11 illustrates an absorbent article comprising one transverse axis.

FIG. 12 illustrates an absorbent article folded about one transverse axis.

FIG. 13 illustrates an absorbent article folded about two transverse axes.

FIG. 14 is a cut-away view of an absorbent article.

FIG. 15 is a cross-sectional view of Section A-A′ of FIG. 14.

FIG. 16 is a cross-sectional view of a tri-folded absorbent article comprising an adhesive fastener in a pouch with a flap.

FIG. 17 is a cross-sectional view of a tri-folded absorbent article comprising an adhesive fastener and a release strip in a pouch with a flap.

FIG. 18 illustrates a bi-fold arrangement in which the pouch comprises a flap panel in which the first absorbent article section is releasably attached to the first panel inwardly oriented face and the second absorbent article section is releasably attached to the second panel inwardly oriented face.

FIG. 19 illustrates a bi-fold arrangement in which the pouch comprises a flap panel in which the first absorbent article section is releasably attached to the second panel inwardly oriented face and the second absorbent article section is releasably attached to the flap panel inwardly oriented face.

FIG. 20 illustrates a bi-fold arrangement in which the pouch does not comprise a flap panel and the first absorbent article section is releasably attached to the first panel inwardly oriented face, the second absorbent article section is releasably attached to the second panel inwardly oriented face.

FIG. 21 is a cut-away view illustrating an embodiment in which the container contains between about 30 and about 100 pouches arranged in two stacks, each stack comprising an approximately equal number of pouches.

### DETAILED DESCRIPTION OF THE INVENTION

As used herein the term “joined” refers to the condition where a first member is connected to a second member either directly or indirectly. Where the first member is connected to an intermediate member, which in turn is connected to the second member, the first member and second member are joined indirectly.

Attached refers to the condition in which a first member may be separated from a second member at least once and one or more of the members is destroyed, distorted, or otherwise marred when the first member is separated from the second member.

Releasably attached refers to the condition in which a first member may be separated from a second member at least once without causing destruction, undue distortion, or undue marring of either member.

Line, as used herein, refers to a straight line, a curved line, or a line that is a combination of straight and curved portions.

Sanitary napkin, as used herein, refers to a device to be used by a menstruating woman.

Panti-liner, as used herein, refers to a device to be used by a woman to absorb vaginal discharge or light menstruation.

Incontinence device, as used herein, refers to an absorbent article to be used by a person having limited bowel or bladder control.

Stacked, as used herein, refers to an arrangement of items in which the articles are arranged in a substantially facing relationship with one another in any orientation.

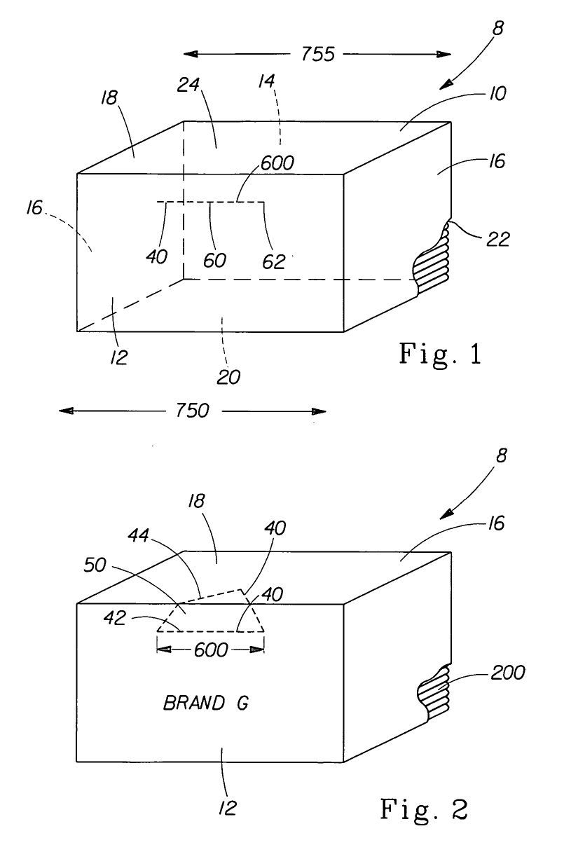

FIG. 1 illustrates a package of absorbent articles 8. The package of absorbent articles 8 comprises a container 10. The container 10 includes a front panel 12, a back panel 14 opposing the front panel 12, opposing side panels 16, a top panel 18, and a bottom panel 20 opposing the top panel 18, which are joined together to form an interior compartment 22. Front panel 12 refers to the panel of the container generally intended to face the user when the user is retrieving an absorbent article from the container 10.

The container 10 may be comprised of a flexible sheet material 24 or any other suitable material. Flexible sheet materials 24 are materials that are conformable to irregularly shaped surfaces. Flexible sheet materials 24 can be materials including, but not limited to, plastic, plastic film, fabric, paper, polymer films, non-woven webs or fabrics, and woven webs or fabrics. The flexible sheet materials 24 can be blown or cast film materials in a blend of low density polyethylene and linear low density polyethylene, metallocenes, ethylene vinyl acetate, SURLYN, polyethylene terephthalate, biaxially oriented polypropylene, and nylon. The flexible sheet material 24 may comprise two or more flexible sheet materials laminated together. One suitable flexible sheet material 24 is Exopack 1020 film available from Exopack LLC, Spartanburg, S.C.

The panels of the container 10 can be joined by means known in the art of making containers from flexible sheet materials 24 such as adhesive bonding, thermal bonding, ultrasonic bonding, and the like. The panels may be comprised of a single sheet of flexible sheet material 24 that is formed into a gusseted container, which yields a container having substantially flat panels. Other methods suitable for forming the container 10 from a single sheet of flexible sheet material 24 can also be used. Container 10 can be made by methods known in the art, including “flow wrap” processes in which a web of material is conveyed, cut, folded, seamed, filled, and closed.

As shown in FIG. 1, the package of absorbent articles 8 comprises a line of weakness 40. The line of weakness 40 can formed by perforating the flexible sheet material 24 or by weakening the flexible sheet material 24 along a prescribed line using other means well known in the art such that the line of weakness 40 is frangible. At least a portion of the line of weakness 40 may be located on the front panel 12. In certain embodiments, the line of weakness 40 can be located entirely on the front panel 12. As shown in FIG. 2, a portion of the line of weakness 40 can be located on the top panel 18. The portion of the line of weakness 40 located on the front panel 12 can have a front panel line of weakness length 42 defined by the length of line of weakness 40 located on the front panel 12. The portion of the line of weakness 40 located on the top panel 18 can have a top panel line of weakness length 44 defined by the length of the line of weakness 40 located on the top panel 18. The front panel line of weakness length 42 can be about the same as the top panel line of weakness length 44 or the front panel line of weakness length 42 can be different from the top panel line of weakness length 44.

The line of weakness 40 can define a predetermined portion 50 of the container 10 that can be separated from the container 10. The predetermined portion 50 can have an opening dimension 600, wherein the opening dimension 600 is the maximum dimension of the predetermined portion 50. The predetermined portion 50 can have a substantially circular shape when flattened. Alternatively, the predetermined portion 50, when flattened, can be substantially oval shaped, a polygon, or an irregular shape having curved edges or a combination of straight edges and curved edges. The predetermined portion 50 can be almost any shape. For a predetermined portion 50 that when flattened has a substantially circular shape, the opening dimension 600 is the diameter of the substantially circular shape. For a predetermined portion 50 that when flattened has a polygonal shape, the opening dimension 600 is the length between the two vertices that are farthest from one another. For an irregularly shaped predetermined portion 50, the opening dimension 600 is the maximum dimension of the predetermined portion 50.

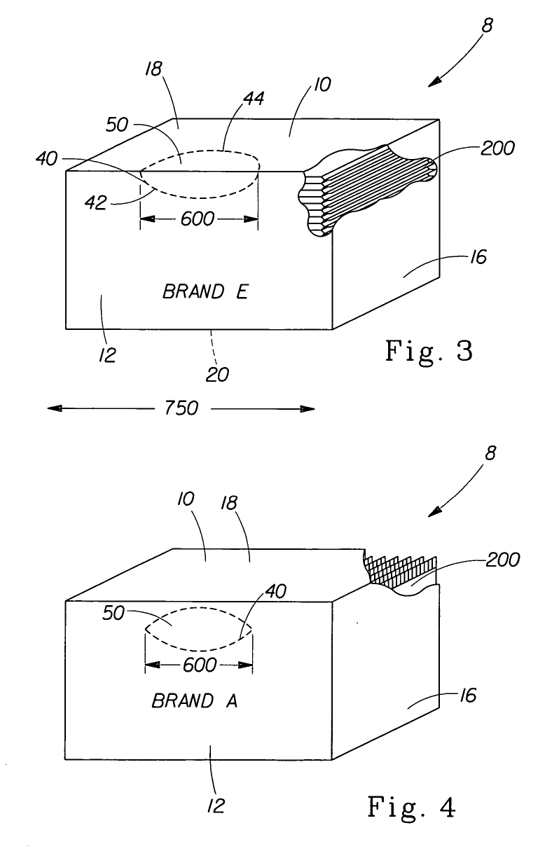

As shown in FIGS. 2 and 3, the predetermined portion 50 can reside partially in the front panel 12 and partially in the top panel 18. FIGS. 2 and 3 illustrate a container 10 in which the line of weakness 40 is partially located in the front panel 12 and partially located in the top panel 18. The line of weakness 40, in FIG. 3, defines a predetermined portion 50 that when flattened comprises a substantially circular shape. In an alternative embodiment shown in FIG. 4, the predetermined portion 50 can reside entirely on the front panel 12 of the container 10.

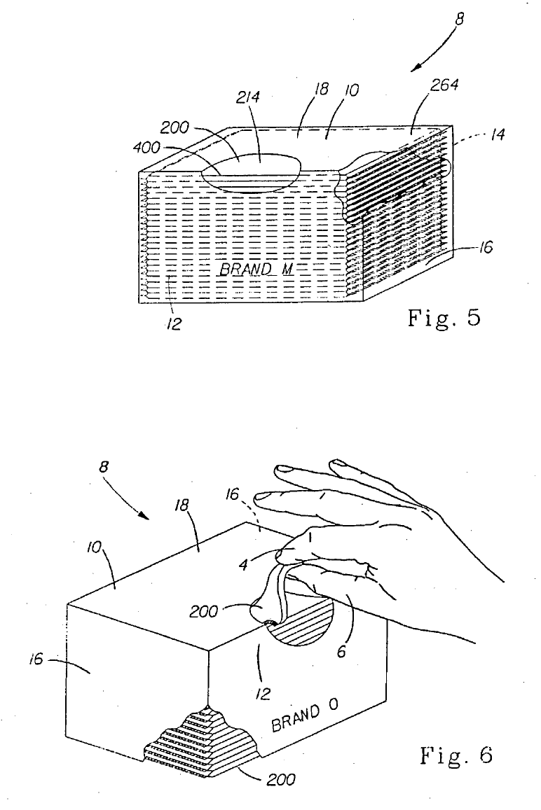

FIG. 5 illustrates a container 10 in which the predetermined portion 50 is separated from the container 10, thereby leaving an opening through which a pouch 200, which contains at least one absorbent article, can be retrieved. In the embodiment of the package of absorbent articles 8 shown in FIG. 5, the predetermined portion 50 is substantially circular, but is not shown because FIG. 5 illustrates the container after the consumer has separated the predetermined portion 50 from the container 10. As shown in FIG. 6, the consumer uses her thumb 6 and forefinger 4 of one hand to grasp a pouch 200. The consumer pulls the pouch 200 from the container 10 through the hole in the container 10 created when the consumer separates the predetermined portion 50 from the container 10. To resist the pulling force, the consumer typically grasps the side panels 16 with the thumb and forefinger of her other hand and perhaps cups the bottom panel 20 of the container in the palm of the same hand. The opening dimension 600 need only be large enough to permit the consumer's thumb 6 and forefinger 4 to fit into the container easily and comfortably.

As shown in FIG. 1, the line of weakness 40 can define an opening slit 60 having an opening dimension 600. For an opening slit 60, the opening dimension 600 is the length of the line of weakness 40.

The opening dimension 600 can be about half the top panel width 755 and about half the front panel width 750. The opening dimension can 600 can be about two-thirds of the top panel width 755 and about two-thirds the front panel width 750. The opening dimension 600 can be less than about half the top panel width 755 and less than about half the front panel width 750.

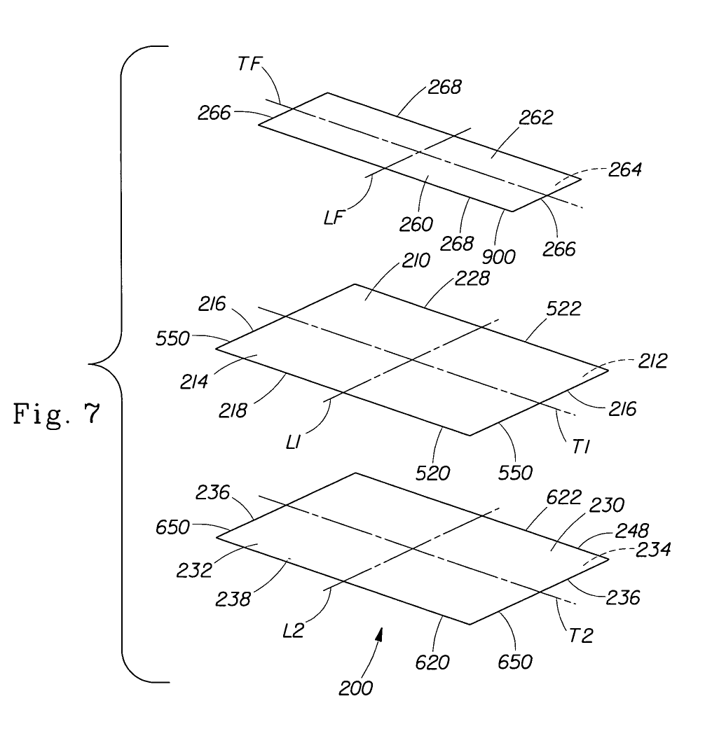

FIG. 7 illustrates the components of an embodiment of pouch 200. The pouch 200 can be comprised of a first panel 210 which has a first panel longitudinal centerline L1, a first panel transverse centerline T1 generally orthogonal to the first panel longitudinal centerline L1 and in plane with the first panel 210, a first panel inwardly oriented face 212, and a first panel outwardly oriented face 214 opposing the first panel inwardly oriented face 212. The first panel 210 has a pair of first panel longitudinal side edges 216 generally disposed along the first panel transverse centerline T1. The first panel 210 has a first panel lower transverse edge 218 generally disposed along the first panel longitudinal centerline L1 and has a first panel lower transverse edge width 520 defined by the distance between the first panel longitudinal side edges 216 along the first panel lower transverse edge 218. The first panel 210 has a first panel upper transverse edge 228 generally disposed along the first panel longitudinal centerline L1 and a first panel upper transverse edge width 522 defined by the distance between the first panel longitudinal side edges 216 along the first panel upper transverse edge 228.

Of the first panel lower transverse edge width 520 and first panel upper transverse edge width 522, at least one can be greater than the opening dimension 600. The first panel lower transverse edge width 520 can be greater than the opening dimension 600. The first panel upper transverse edge width 522 can be greater than the opening dimension 600. The first panel upper transverse edge width 522 and first panel lower transverse edge width 520 can both be greater than the opening dimension 600. The first panel longitudinal side edges 216 have a first panel longitudinal side edge length 550 defined by the distance between the first panel lower transverse edge 218 and the first panel upper transverse edge 228. The first panel longitudinal side edge length 550 can be greater than the opening dimension 600.

The pouch 200, shown in FIG. 7, is further comprised of a second panel 230 and has a second panel longitudinal centerline L2, a second panel transverse centerline T2 generally orthogonal to the second panel longitudinal centerline L2 and in plane with the second panel 230, a second panel inwardly oriented face 232, and a second panel outwardly oriented face 234 opposing the second panel inwardly oriented face 232. The second panel 230 has a pair of second panel longitudinal side edges 236 generally disposed along the second panel transverse centerline T2. The second panel 230 has a second panel lower transverse edge 238 generally disposed along the second panel longitudinal centerline L2 and has a second panel lower transverse edge width 620 defined by the distance between the second panel longitudinal side edges 236 along the second panel lower transverse edge 238. The second panel 230 has a second panel upper transverse edge 248 generally disposed along the second panel longitudinal centerline L2 and a second panel upper transverse edge width 622 defined by the distance between the second panel longitudinal side edges 236 along the second panel upper transverse edge 248.

Of the second panel lower transverse edge width 620 and second panel upper transverse edge width 622, at least one is greater than the opening dimension 600. The second panel lower transverse edge width 620 can be greater than the opening dimension 600. The second panel upper transverse edge width 622 can be greater than the opening dimension 600. The second panel lower transverse edge width 620 and second panel upper transverse edge width 622 can both be greater than the opening dimension 600. The second panel longitudinal side edges 236 have a second panel longitudinal side edge length 650 defined by the distance between the second panel lower transverse edge 238 and the second panel upper transverse edge 248. The second panel longitudinal side edge length 650 can be greater than the opening dimension 600.

The first panel lower transverse edge width 520, first panel upper transverse edge width 522, second panel lower transverse edge width 620, and second panel upper transverse edge width 622 can be more than about one and one half times greater than the opening dimension 600. The first panel longitudinal side edge length 550 and second panel longitudinal side edge length 650 can be more than about one and one half times greater than the opening dimension 600. The first panel lower transverse edge width 520, first panel upper transverse edge width 522, second panel lower transverse edge width 620, and second panel upper transverse edge width 622 can be more than about two times greater than the opening dimension 600.

The pouch 200 may be comprised of any flexible material including, but not limited to, a polymer film, blown or cast film materials in a blend of low density polyethylene and linear low density polyethylene, metallocenes, ethylene vinyl acetate, SURLYN, polyethylene terephthalate, biaxially oriented polypropylene, nylon, a woven fabric, a nonwoven fabric, a composite laminated material, and combinations thereof.

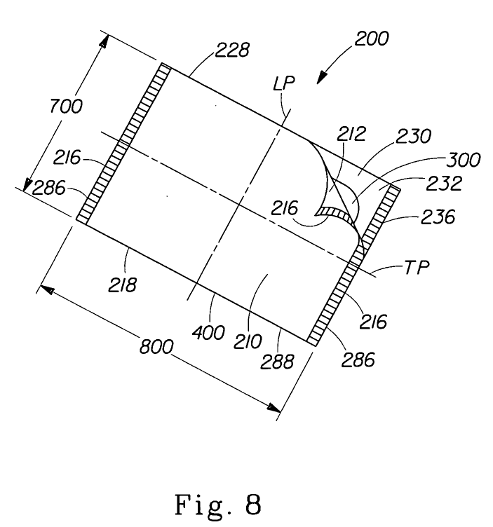

The first panel 210 and second panel 230 may be comprised of a single piece of sheet material. The first panel 210 and second panel 230 may be folded into the configuration shown in FIG. 8, forming fold-line 400. The fold-line 400 can coincide with the first panel lower transverse edge 218 and the second panel lower transverse edge 238. The first panel longitudinal centerline L1 and the second panel longitudinal centerline L2 can be generally perpendicular to the fold-line 400 separating the first panel 210 and second panel 230.

The second panel longitudinal side edges 236 may be releasably attached to the first panel longitudinal side edges 216 such that the second panel inwardly oriented face 232 faces the first panel inwardly oriented face 212. The second panel longitudinal side edges 236 may be releasably attached to the first panel longitudinal side edges 216 by seals including, but not limited to, heat seals, pressure seals, adhesives, glues, ultrasonic seals, or other types of seals.

The pouch 200 has a pouch longitudinal centerline LP and a pouch transverse centerline TP. The pouch longitudinal centerline LP is generally perpendicular to the fold-line 400. The pouch transverse centerline TP is generally orthogonal to the pouch longitudinal centerline LP and in plane with the pouch 200. The pouch 200 has a pair of pouch longitudinal edges 286 generally disposed along the pouch transverse centerline TP. The pouch 200 has a pair of pouch transverse edges 288 generally disposed along the pouch longitudinal centerline LP. The pouch longitudinal edge length 700 is the distance between the pair of pouch transverse edges 288. The pouch transverse edge width 800 is the distance between the pair of pouch longitudinal edges 286.

The pouch longitudinal edge length 700 can be greater than the opening dimension 600. The pouch transverse edge width 800 can be greater than the opening dimension 600. The pouch longitudinal edge length 700 and pouch transverse edge width 800 can both be greater than the opening dimension 600. The pouch transverse edge width 800 can be more than about one and one half times greater than the opening dimension 600. The pouch transverse edge width 800 can be more than about two times greater than the opening dimension 600. The pouch longitudinal edge length 700 can be more than about one and one half times greater than the opening dimension 600. The pouch can be described as having an aspect ratio defined by the pouch longitudinal edge length 700 divided by the pouch transverse edge width 800. In some embodiments, the pouch aspect ratio can be between about 0.5 and about 1.2. In some embodiments, the pouch aspect ratio can be between about 0.75 and about 1.

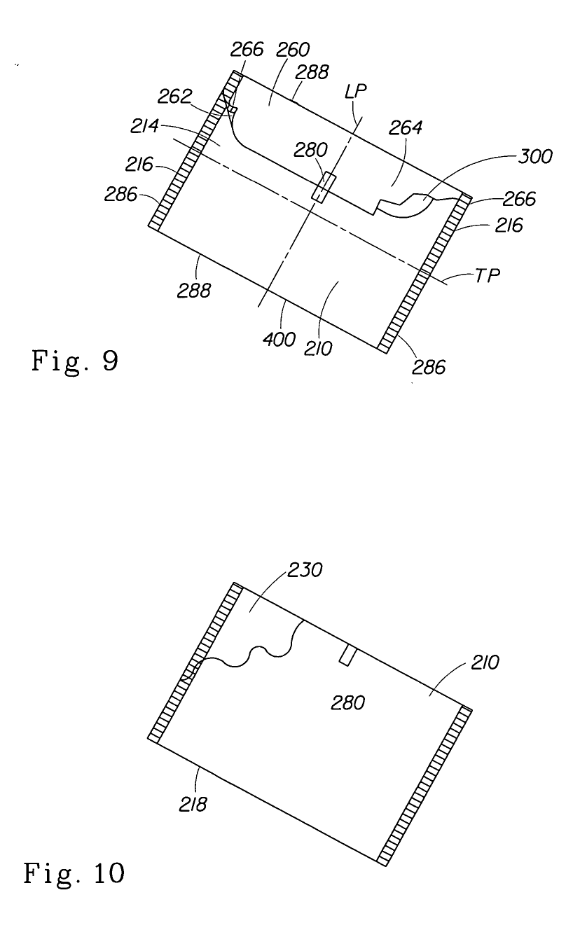

The pouch 200 can further comprise a flap panel 260, as shown in FIG. 9. The flap panel 260 is also shown in FIG. 7, in which the flap panel 260 is shown in an orientation which the flap panel 260 would be joined to the second panel 230. The flap panel 260 has a flap panel longitudinal centerline LF, a flap panel transverse centerline TF generally orthogonal to the flap panel longitudinal centerline LF and in plane with the flap panel 260, a flap panel inwardly oriented face 262, and a flap panel outwardly oriented face 264 opposing the flap panel inwardly oriented face 262. The flap panel longitudinal centerline LF is generally perpendicular to the fold-line 400 separating the first panel 210 and second panel 230.

The flap panel 260 has a pair of flap panel longitudinal side edges 266 generally disposed along the flap panel transverse centerline TF. The flap panel 260 has a flap panel lower transverse edge 268 generally disposed along the flap panel longitudinal centerline LF. The flap panel lower transverse edge 268 can be joined to the second panel upper transverse edge 248 using any method known in the art including, but not limited to, adhesive bonding, thermal bonding, ultrasonic bonding, and the like. The flap panel 260, second panel 230, and first panel 210 can be comprised of a single piece of sheet material that is folded into the configuration shown in FIG. 9.

The flap panel lower transverse edge 268 has a flap panel lower transverse edge width 900 defined by the distance between the flap panel longitudinal side edges 266. The flap panel lower transverse edge width 900 can be greater than the opening dimension 600. The flap panel lower transverse edge width 900 can be more than about one and one half times greater than the opening dimension 600. The flap panel lower transverse edge width 900 can be more than about two times greater than the opening dimension 600.

As shown in FIG. 9, the flap panel longitudinal side edges 266 can be releasably attached to the first panel longitudinal side edges 216 such that the flap panel inwardly oriented face 262 faces the first panel outwardly oriented face 214. The flap panel longitudinal side edges 266 can be releasably attached to the first panel longitudinal side edges 216 by seals including, but not limited to, heat seals, pressure seals, adhesives, glues, ultrasonic seals.

The pouch 200 can also include a pull tab 280. If the pouch 200 does not include a flap panel 260, a portion of the pull tab 280 can be releasably attached to the first panel 210 and a portion of the pull tab 280 can be joined, attached, or releasably attached to the second panel 230, as shown in FIG. 10. Alternatively, the pull tab 280 can be an integral part of second panel 230 such that the pull tab 280 is an extension of the second panel 230. Other configurations for the pull tab 280 which are known in the art can also be used.

If the pouch 200 includes a flap panel 260, a portion of the pull tab 280 can be releasably attached to the first panel 210 and portion of the pull tab 280 can be joined, attached, or releasably attached to the flap panel 260. Alternatively, the pull tab 280 can be an integral part of flap panel 260 such that the pull tab 280 is an extension of the flap panel 260. U.S. Pat. No. 5,569,230 issued to Fisher et al., U.S. Pat. No. 5,792,131 issued to Mizutani, and U.S. Pat. No. 5,683,377 issued to Mizutani, disclose pouches comprising a pull tab 280.

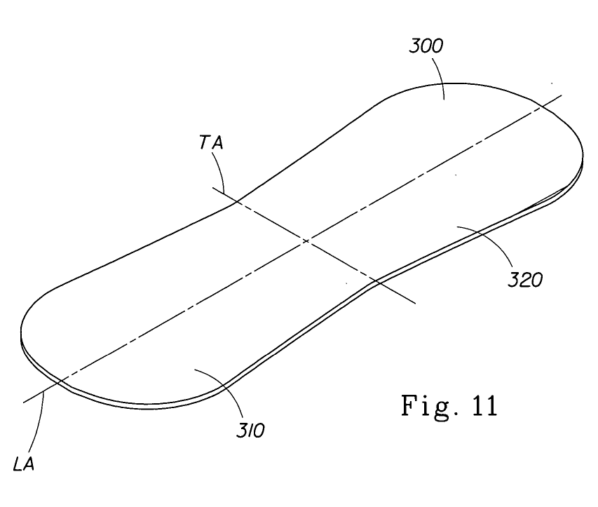

An example of an embodiment of absorbent article 300 is shown in FIG. 11. The absorbent article can be a sanitary napkin, a panti-liner, an incontinence product, or other similar absorbent article to be worn in the crotch of the wearer. The absorbent article 300 has a longitudinal centerline LA and one or more absorbent article transverse axes TA. The term longitudinal, as used herein with respect to an absorbent article 300, refers to a line, axis or direction in the plane of the absorbent article 300 that is generally aligned with (e.g., substantially parallel to) a vertical plane which bisects a standing wearer into left and right body halves when the absorbent article 300 is worn in the wearer's crotch. The term transverse, with respect to an absorbent article 300, refers to a line, axis or direction which lies within the plane of the absorbent article 300 that is generally perpendicular to the longitudinal direction.

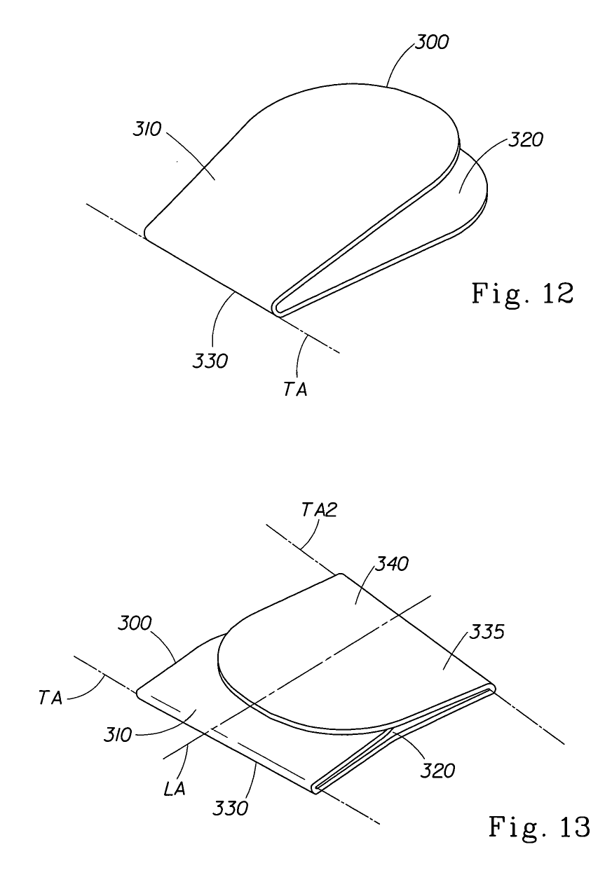

If the absorbent article 300 has one absorbent article transverse axis TA, the absorbent article 300 can be folded about the absorbent article transverse axis TA to form a first fold-line 330, as shown in FIG. 12. The first fold-line 330 divides the absorbent article 300 into a first absorbent article section 310 and a second absorbent article section 320 and the first fold-line 330 defines the boundary between the first absorbent article section 310 and the second absorbent article section 320. A configuration in which absorbent article 300 has one absorbent article transverse axis TA will be referred to herein as a “bi-fold” arrangement.

As shown in FIG. 13, if absorbent article 300 has a second absorbent article transverse axis TA2, the absorbent article 300 can be folded about the absorbent article transverse axis TA to form a first fold-line 330 and folded about the second absorbent article transverse axis TA2 to form a second fold-line 335. The first fold-line 330 defines a boundary between the first absorbent article section 310 and the second absorbent article section 320. The second fold-line 335 defines a boundary between the second absorbent article section 320 and the third absorbent article section 340. A configuration in which absorbent article 300 is folded about the absorbent article transverse axis TA and folded about the second absorbent article transverse axis TA2 will be referred to herein as a “tri-fold” arrangement.

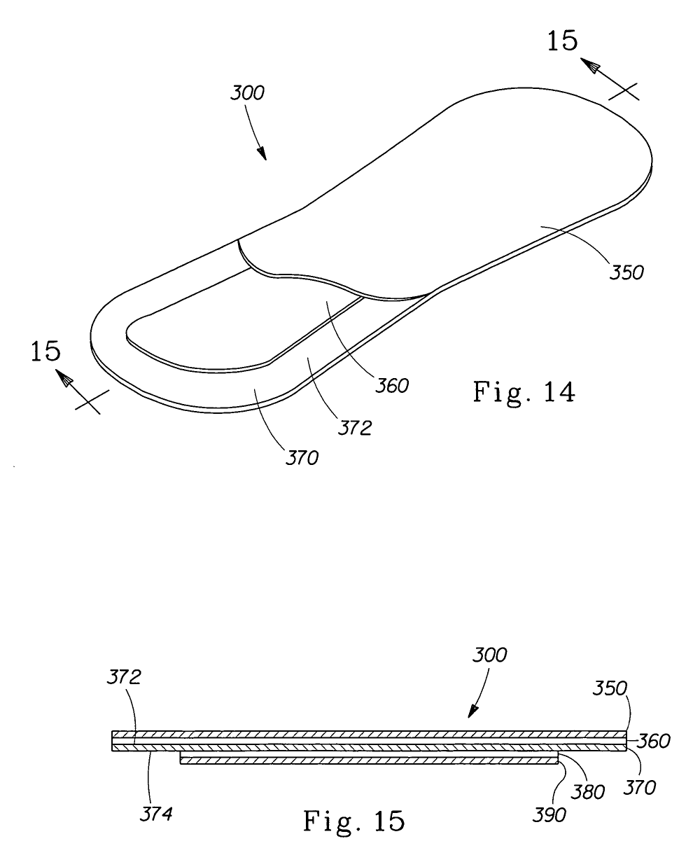

An absorbent article 300 comprised of multiple components is shown in FIG. 14. Absorbent article 300 can comprise a liquid pervious topsheet 350 and a backsheet 370 joined to the topsheet 350. The backsheet 370 has a backsheet inwardly oriented face 372 and a backsheet outwardly oriented face 374 (shown in FIG. 15) opposing the backsheet inwardly oriented face 372. The absorbent article 300 can further comprise an absorbent core 360 positioned between the topsheet 350 and the backsheet 370.

One suitable liquid pervious topsheet 350 is an apertured polymer film sold under the trade name DRI-WEAVE® by the The Procter and Gamble Co., Cincinnati, Ohio. Another suitable liquid pervious topsheet 350 is disclosed in U.S. Pat. No. 4,629,643 and can be obtained from Tredegar Film Products, Terre Haute, Ind. under the designation X27121. The absorbent core 360 can be Foley Fluff pulp available from Buckeye Technologies Inc., Memphis, Tenn., that is disintegrated and formed into a core having a density of about 0.07 grams per cubic centimeter (g/cm³) and a caliper of about 3 mm. The backsheet 370 can be any suitable flexible material and is generally a liquid impervious or liquid resistant material, such as a polyolefinic film.

As shown in FIG. 15, absorbent article 300 can further comprise an adhesive fastener 380. The adhesive fastener 380 can be on the backsheet outwardly oriented face 374, as shown in FIG. 15. The adhesive fastener 380 can be hot-melt adhesive material capable of establishing a temporary bond with the wearer's panty. A suitable material is the composition designated HL-1491 XZP, commercially available from H. B. Fuller, Toronto, Ontario, Canada.

Absorbent article 300 can also comprise a release strip 390 that is releasably attached to the adhesive fastener 380. The release strip 390 can be made of film, kraft paper, calendered paper, or other materials known in the art. The side of the release strip 390 facing the adhesive fastener can be coated with a release coating such as silicone. Examples of a suitable release strip 390 include “SILOX” 4R/O and Silox CIS, marketed by Akrosil, Menasha, Wis.

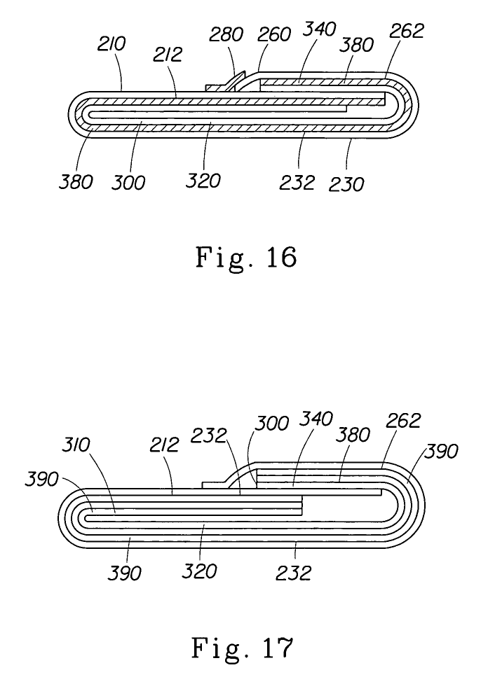

Absorbent article 300 can be placed in the pouch 200 in a variety of orientations. FIG. 16 illustrates a tri-fold arrangement in which the absorbent article 300 comprises an adhesive fastener 380 in which the adhesive fastener 380 located within the first absorbent article section 310 is releasably attached to the first panel inwardly oriented face 212, adhesive fastener 380 located within the second absorbent article section 320 is releasably attached to the second panel inwardly oriented face 232, and the adhesive fastener 380 located within the third absorbent article section 340 is releasably attached to the flap panel inwardly oriented face 262.

With respect to the describing the absorbent article 300 as being in a pouch 200, the absorbent article 300 is considered to be in a pouch 200 when a portion of the absorbent article 300 or the entire absorbent article 300 is between the first panel 210 and second panel 230. The absorbent article 300 can be oriented in the pouch 200 such that the absorbent article transverse axis TA is substantially parallel to the pouch transverse centerline TP or the pouch longitudinal centerline LP. The absorbent article 300 can be oriented in the pouch 200 such that the second absorbent article transverse axis TA2, if present, is substantially parallel to the pouch transverse centerline TP or the pouch longitudinal centerline LP.

FIG. 17 illustrates a cross-sectional view of a tri-fold arrangement in which the absorbent article 300 comprises an adhesive fastener 380 and a release strip 390 in which the release strip 390 located within the first absorbent article section 310 is attached to the first panel inwardly oriented face 212, release strip 390 located within the second absorbent article section 320 is attached to the second panel inwardly oriented face 232, and release strip 390 located within the third absorbent article section 340 is attached to the flap panel inwardly oriented face 262. Alternatively, release strip 390 located within the first absorbent article section 310 can be releasably attached or joined to the first panel inwardly oriented face 212, release strip 390 located within the second absorbent article section 320 can be releasably attached or joined to the second panel inwardly oriented face 232, and release strip 390 located within the third absorbent article section 340 can be releasably attached or joined to the flap panel inwardly oriented face 262. An adhesive can be used to attach, releasably attach, or join the release strip 390 to components of the pouch 200. The adhesive for attaching, releasably attaching, or joining the release strip 390 to components of the pouch can have a peel force that is greater than the peel force of the adhesive fastener 380.

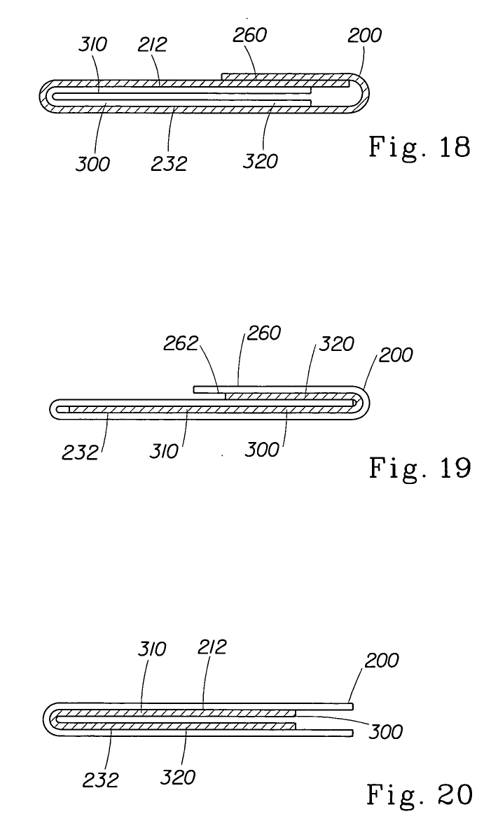

FIG. 18 illustrates a cross-sectional view of a bi-fold arrangement in which the pouch 200 comprises a flap panel 260. Absorbent article 300 is bi-folded and the first absorbent article section 310 is releasably attached to the first panel inwardly oriented face 212. The second absorbent article section 320 can also be releasably attached to the second panel inwardly oriented face 232. The first absorbent article section 310 and second absorbent article section 320 do not both have to be releasably attached to a panel of the pouch 200.

FIG. 19 illustrates a cross-sectional view of a bi-fold arrangement in which the pouch 200 comprises a flap panel 260, absorbent article 300 is bi-folded, and the first absorbent article section 310 is releasably attached to the second panel inwardly oriented face 232. The second absorbent article section 320 can also be releasably attached to the flap panel inwardly oriented face 262. The first absorbent article section 310 and second absorbent article section 320 do not both have to be releasably attached to a panel of the pouch 200.

Bi-fold arrangements like those illustrated in FIGS. 18 and 19 can also be operative if the absorbent article 300 further comprises an adhesive fastener 380. The absorbent article 300 can also further comprises a release strip 390 and a portion or all of the release strip 390 can be attached, releasably attached, or joined to one or more panels of the pouch 200 in configurations like those shown in FIGS. 18 and 19.

FIG. 20 illustrates a bi-fold arrangement in which the pouch 200 does not comprise a flap panel 260. The first absorbent article section 310 can be releasably attached to the first panel inwardly oriented face 212 and the second absorbent article section 320 can be releasably attached to the second panel inwardly oriented face 232. As discussed previously, absorbent article 300 can further comprise an adhesive fastener 380. The adhesive fastener 380 can be releasably attached to the first panel inwardly oriented face 212 and/or the second panel inwardly oriented face 232 in an arrangement like that shown in FIG. 20. The absorbent article 300 can further comprise a release strip 390. The release strip 390 can be attached, releasably attached, or joined to the first panel inwardly oriented face 212 and/or the second panel inwardly oriented face 232 in an arrangement like that shown in FIG. 20.

The pouch 200 can be placed within the container 10 such that one of the pouch transverse edges 288 is substantially facing the front panel 12. The pouch 200 can be placed within the container 10 such that one of the pouch transverse edges 288 is substantially facing the top panel 18. The pouch 200 can be placed within the container 10 such that one of the pouch transverse edges 288 is substantially facing the front panel 12 and substantially parallel to the top panel 18. The pouch 200 can be placed within the container 10 such that one of the pouch transverse edges 288 is substantially facing the top panel 18 and substantially parallel to the front panel 12.

Without being bound by theory, it is believed that the package of absorbent articles 8 disclosed herein presents a number of benefits to the consumer. The line of weakness 40 allows the consumer to open the package of absorbent articles 8 in a controlled manner without uncontrollable or irregular distortion and tearing of the container 10. The pouch 200 serves to protect the absorbent article 300 while the absorbent article 300 is stored in an open container 10 and after the consumer removes the absorbent article 300 from the container 10.

By making the pouch longitudinal edge length 700 and pouch transverse edge width 800 greater than the opening dimension 600, the pouches 200 are unlikely to unintentionally fall out of the container 10.

Although a pouch 200 containing an unfolded absorbent article 300 might protect the absorbent article 300, a folded absorbent article 300 makes the pouch 200 bulkier, which allows the consumer to more easily retrieve the pouch 200 from the container 10. By orienting the pouch 200 within the container 10 as disclosed herein, it is believed that a relatively thick and rounded edge of the pouch is presented to the consumer through the opening created when the consumer separates the predetermined portion 50 from the container 10. Thus, the consumer can easily insert her thumb 6 and forefinger 4 into the container 10 and grasp a single pouch 200, as shown in FIG. 6.

Folding the absorbent article 300 also permits the largest dimension of the container 10 to be made smaller, which allows the consumer to handle the container 10 with one hand easily. Folding the absorbent article 300 twice can permit an even smaller container 10 to be used. Absorbent articles 300 such as sanitary napkins, panti-liners, and incontinence products typically are between about 120 mm and about 280 mm long and about 40 mm to about 80 mm wide. Thus, a bi-folded absorbent article 300 can be between about 60 mm to about 140 mm long and a tri-folded absorbent article 300 can be between about 40 mm and about 95 mm long. A tri-folded absorbent article 300 can be folded more compactly than a bi-folded absorbent article 300.

The dimensions of the pouch 200 need only to be slightly larger than the folded absorbent article 300 contained therein. As the consumer pulls the pouch 200 through the opening created when the consumer separates the predetermined portion 50 from the container 10 or the opening slit 60, the pouch 200 and absorbent article 300 contained therein offer resistance to being pulled though the opening. For the pouch 200 to fit through the opening or opening slit 60, the pouch 200 and absorbent article 300 contained therein deforms. As shown in FIG. 6, the deformed shape can be an arch-like shape that roughly conforms to the shape of a portion of the opening created when the consumer separates the predetermined portion 50 from the container 10. The deformed shape can be other shapes depending on the geometry of the predetermined portion 50 or opening slit 60. The shape of the opening created when the consumer separates the predetermined portion 50 from the container 10 can also deform as the consumer pulls the pouch 200 through the opening because the container 10 is comprised of flexible sheet material 24. The pouch 200 will tend to flatten out once the consumer completely withdraws the pouch 200 from the container 10 and the shape of the container 10 will rebound to the shape that the container 10 had prior to removing a pouch 200.

The container 10 can contain any number of pouches 200 or absorbent articles 300. A container 10 having a substantially rectangular parallelepiped shape can be easily handled by the consumer. A substantially cubic shaped container 10 can be a useful configuration. In some embodiments, the container 10 may contain between about 10 and about 40 pouches with each pouch containing at least one absorbent article 300 that is tri-folded. In some embodiments, a substantially palm sized container is convenient for the user to handle. The bottom panel 20 can have a surface area that is less than about 7300 square millimeters and the largest edge length of the bottom panel can be less than about 90 mm.

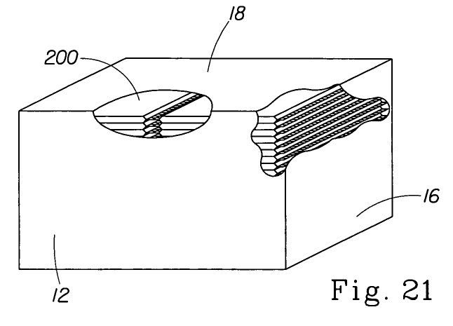

A container 10 containing 30 pouches in a single stack with the absorbent articles 300 tri-folded can be suitable. In an alternative embodiment, the container 10 can contain between about 30 and about 100 pouches 200 in which the absorbent articles 300 are tri-folded and the pouches 200 can be arranged in two or more stacks, each comprising an approximately equal number of pouches, as shown in FIG. 21.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A package of absorbent articles, said package comprising:
a container comprising a front panel, a back panel opposing said front panel, opposing side panels, a top panel, and a bottom panel opposing said top panel, which are joined together to form an interior compartment, wherein said front panel, said back panel, said opposing side panels, said top panel, and said bottom panel comprise flexible sheet material;
a line of weakness, wherein at least a portion of said line of weakness is located within said front panel, wherein said line of weakness defines a predetermined portion to be separated from said container, wherein said predetermined portion comprises an opening dimension;
a plurality of pouches arranged in a stacked relationship in said interior compartment of said container, each said pouch comprising a pouch longitudinal centerline and a pouch transverse centerline, a pair of pouch longitudinal edges generally disposed along said pouch transverse centerline and a pair of pouch transverse edges generally disposed along said pouch longitudinal centerline, a pouch longitudinal edge length defined by the distance between said pair of pouch transverse edges, a pouch transverse edge width defined by the distance between said pair of pouch longitudinal edges, wherein said pouch longitudinal edge length and at least one said pouch transverse edge width are greater than said opening dimension; and
a plurality of folded absorbent articles, wherein at least one folded absorbent article is contained in each pouch.

2. The package of absorbent articles according to claim 1 wherein a portion of said line of weakness is located within said top panel.

3. The package of absorbent articles according to claim 1 wherein said pouch transverse edge width is more than about two times greater than said opening dimension.

4. The package of absorbent articles according to claim 1 wherein one of said pouch transverse edges is substantially facing said front panel and substantially parallel to said top panel.

5. The package of absorbent articles according to claim 1 wherein one of said pouch transverse edges is substantially facing said top panel and substantially parallel to said front panel.

6. The package of absorbent articles according to claim 1 wherein said folded absorbent article is bi-folded or tri-folded.

7. The package of absorbent articles according to claim 1 wherein said pouch has an aspect ratio defined by the pouch longitudinal edge length divided by the pouch transverse edge width, wherein said aspect ratio is between about 0.5 and about 1.2.

8. The package of absorbent articles according to claim 1 wherein said container contains between about 10 and about 40 pouches arranged in a single stack.

9. The package of absorbent articles according to claim 1 wherein said container contains between about 30 and about 100 pouches arranged in two or more stacks, each said stack comprising an approximately equal number of pouches.

10. The package of absorbent articles according to claim 1 wherein said absorbent articles are selected from the group consisting of sanitary napkins, panti-liners, and incontinence products.

11. The package of absorbent articles according to claim 1 wherein said bottom panel has a bottom panel surface area and a bottom panel largest edge length, wherein said bottom panel surface area is less than about 7300 square millimeters and said bottom panel largest edge length is less than about 90 mm.

12. A package of absorbent articles, said package comprising:
a container comprising a front panel having a front panel width, a back panel opposing said front panel, opposing side panels, a top panel having a top panel width, and a bottom panel opposing said top panel, which are joined together to form an interior compartment, wherein said front panel, said back panel, said opposing side panels, said top panel, and said bottom panel comprise flexible sheet material;
a line of weakness, wherein a portion of said line of weakness is located within said front panel and a portion of said line of weakness is located within said top panel, wherein said portion of said line of weakness located within said front panel has a front panel line of weakness length and said portion of said line of weakness located within said top panel has a top panel line of weakness length and said front panel line of weakness length is about the same as said top panel line of weakness length, wherein said line of weakness defines a predetermined portion to be separated from said container, wherein said predetermined portion when separated from said container and flattened comprises a substantially circular shape, wherein said predetermined portion comprises an opening dimension, wherein said opening dimension is about half said top panel width and about half said front panel width;
a plurality of pouches arranged in a stacked relationship in said interior compartment of said container, each said pouch comprising a pouch longitudinal centerline and a pouch transverse centerline, a pair of pouch longitudinal edges generally disposed along said pouch transverse centerline and a pair of pouch transverse edges generally disposed along said pouch longitudinal centerline, a pouch longitudinal edge length defined by the distance between said pair of pouch transverse edges, a pouch transverse edge width defined by the distance between said pair of pouch longitudinal edges, wherein said pouch longitudinal edge length is greater than said opening dimension and said pouch transverse edge width is about two times greater than said opening dimension, wherein said pouch has an aspect ratio defined by the pouch longitudinal edge length divided by the pouch transverse edge width, wherein said aspect ratio is between about 0.5 and about 1.2; and
a plurality of folded absorbent articles, wherein at least one folded absorbent article is contained in each pouch, wherein said folded absorbent article is tri-folded, wherein said folded absorbent article comprises a liquid pervious topsheet, a backsheet joined to said topsheet, and an absorbent core positioned between said topsheet and said backsheet.

13. A package of absorbent articles, said package comprising:
a container comprising a front panel, a back panel opposing said front panel, opposing side panels, a top panel, and a bottom panel opposing said top panel, which are joined together to form an interior compartment, wherein said front panel, said back panel, said opposing side panels, said top panel, and said bottom panel comprise flexible sheet material;
a line of weakness, wherein said line of weakness is located within said front panel and has a front panel line of weakness length within said front panel, wherein said line of weakness defines an opening slit, wherein said opening slit comprises an opening dimension;
a plurality of pouches arranged in a stacked relationship in said interior compartment of said container, each said pouch comprising a pouch longitudinal centerline and a pouch transverse centerline, a pair of pouch longitudinal edges generally disposed along said pouch transverse centerline and a pair of pouch transverse edges generally disposed along said pouch longitudinal centerline, a pouch longitudinal edge length defined by the distance between said pair of pouch transverse edges, a pouch transverse edge width defined by the distance between said pair of pouch longitudinal edges, wherein said pouch longitudinal edge length and at least one said pouch transverse edge width are greater than said opening dimension; and
a plurality of folded absorbent articles, wherein at least one folded absorbent article is contained in each pouch.

14. The package of absorbent articles according to claim 13 wherein said pouch transverse edge width is more than about two times greater than said opening dimension.

15. The package of absorbent articles according to claim 13 wherein one of said pouch transverse edges is substantially facing said front panel.

16. The package of absorbent articles according to claim 13 wherein said folded absorbent article is bi-folded or tri-folded.

17. The package of absorbent articles according to claim 13 wherein said pouch has an aspect ratio defined by the pouch longitudinal edge length divided by the pouch transverse edge width, wherein said aspect ratio is between about 0.5 and about 1.2.

18. The package of absorbent articles according to claim 13 wherein said container contains between about 10 and about 40 pouches arranged in a single stack.

19. The package of absorbent articles according to claim 13 wherein said container contains between about 30 and about 100 pouches arranged in two or more stacks, each said stack comprising an approximately equal number of pouches.

20. The package of absorbent articles according to claim 13 wherein said absorbent articles are selected from the group consisting of sanitary napkins, panti-liners, and incontinence products.
